(19) United States
(12) Patent Application Publication
(10) Pub. No.: US 2025/0108034 A1
(43) Pub. Date: Apr. 3, 2025

(54) EYE DROP FORMULATION

(71) Applicants: SEMMELWEIS EGYETEM, Budapest (HU); SZEGEDI TUDOMÁNYEGYETEM, Szeged (HU)

(72) Inventors: György Tibor BALOGH, Szeged (HU); Gábor KATONA, Szeged (HU); Ildikó CSÓKA, Szeged (HU); István ZUPKÓ, Szeged (HU); Zoltán Zsolt NAGY, Budapest (HU); Huba KISS, Budapest (HU); Ágnes TAKÁCS, Budapest (HU); Anita CSORBA, Budapest (HU)

(21) Appl. No.: 18/725,516

(22) PCT Filed: Dec. 21, 2022
(86) PCT No.: PCT/HU2022/050090

§ 371 Date: Jun. 28, 2024

(30) Foreign Application Priority Data
Dec. 28, 2021 (HU) P2100456

(51) Int. Cl. A61K 31/365; A61K 9/00; A61K 9/08; A61K 47/18; A61K 47/69; A61P 27/02

(52) U.S. Cl. CPC A61K 31/365; A61K 9/0048; A61K 9/08; A61K 47/186; A61K 47/6951; A61P 27/02

(57) ABSTRACT

An ocular formulation comprising L-Ascorbic acid 6-palmitate (ASP) is provided. The formulation is useful in situations wherein the maintenance of corneal transparency is at risk, e.g. during or after corneal surgeries.

### FIELD OF THE INVENTION

An ocular formulation comprising L-Ascorbic acid 6-palmitate (ASP) is provided. The formulation is useful in situations wherein the maintenance of corneal transparency is at risk, e.g. during or after corneal surgeries.

### BACKGROUND OF THE INVENTION

Corneal transparency is maintained by highly organized composition of collagen fibrils in the stroma (1). Common corneal surgeries, such as cross-linking (CXL) or excimer laser photoablation may change the structure of corneal layers, and lead to the cascade of corneal haze formation. Remodelling process of corneal tissues during wound healing include keratocyte apoptosis, swelling of stromal matrix, and production of less organized collagen fibrils (2). Corneal haze induces increased light scattering and consequent loss of transparency, which may result in complaints as glare, halo or decreased contrast sensitivity and visual quality (3). Usually, the amount of corneal haze is the highest in the early postoperative period, however, significant increasement may be presented one year after the treatment as well (4). Remodelling process during the wound healing response also occurs in other corneal pathologies, such as after thermal or chemical injuries, or infectious keratitis, and may lead to the development of permanent corneal opacifications. Corneal pathologies with opacification may have a significant burden on the vision-related quality of life in concerned patients (5). Commonly, application of corticosteroid-eye drops, and mitomycin-C is used as topical medications in the treatment of corneal opacities, which might be associated with severe long-term complications, such as development of cataract, secondary glaucoma, scleromalacia or perforation (5).

L-Ascorbic acid (AA, ascorbic acid, or vitamin C), an essential water-soluble vitamin has a pivotal role in several physiologic and metabolic functions in the human body. AA has major antioxidant properties, and it is necessary for the biosynthesis of collagen fibrils since it is a required co-factor in hydroxylation. Lack of AA may lead to the production of structurally unstable collagen molecules; thus, it may influence the healing process of the tissues (6). Intravenously administered AA has been shown to be safely and effectively decreased the size of the epithelial defects and the size of the corneal opacity in the treatment of infectious keratitis (13). Antioxidant effects of AA can reduce corneal neovascularization and postoperative stromal opacification after excimer photoablation (14, 15). Stojanovic and Ringvold found that the severity of corneal haze after PRK treatment was significantly reduced in the AA-treated group (16). Therefore, according to the recently available data, AA might have beneficial effects on corneal haze formation. However, the permeability of AA through corneal tissues might be insufficient, since the cornea represents a mainly lipophilic diffusion barrier against hydrophilic agents, such as AA. There is evidence showing that increasing concentration of aqueous solution of free-form AA leads to increased amount of penetrated AA, until a certain point (17).

A composition which effectively aids the corneal wound healing process without side effects is still needed.

### SHORT DESCRIPTION OF THE INVENTION

A pharmaceutical composition is provided, comprising or consisting of an ester of ascorbic acid with a fatty acid, a beta-cyclodextrin substituted with C1-3 alkyl and/or C1-3 hydroxyalkyl, isotonic saline and optionally a preservative.

Preferably the ester of ascorbic acid with a fatty acid is selected from ascorbyl laureate, ascorbyl myristate, ascorbyl stearate, ascorbyl palmitate, ascorbyl oleate, ascorbyl linolate and any mixture thereof. Highly preferably the ester of ascorbic acid with a fatty acid is L-Ascorbic acid 6-palmitate (ASP).

Preferably the beta-cyclodextrin substituted with C1-3 alkyl is methyl-beta-cyclodextrin (random methylated beta-cyclodextrin; RAMEB).

Preferably the beta-cyclodextrin substituted C₁₋₃ hydroxyalkyl is (2-Hydroxypropyl)-beta-cyclodextrin (HPBCD).

Preferably the preservative is benzalkonium chloride (BC).

A pharmaceutical composition is provided, comprising or consisting of ASP, a beta-cyclodextrin selected from RAMEB, HPBCD and mixtures thereof, isotonic saline and optionally BC.

Preferably the pharmaceutical composition consists of ASP, a beta-cyclodextrin selected from RAMEB, HPBCD and mixtures thereof, isotonic saline and BC.

Preferably the pharmaceutical composition comprises or consists of
about 800-1200 μM ASP, preferably about 850-1150 μM, preferably about 900-1100 μM, preferably about 950-1050 μM, highly preferably about 1000 μM ASP, and
about 5-30 mM RAMEB, preferably about 8-25 mM, more preferably about 9-22 mM RAMEB, or
about 8-12 mM RAMEB, preferably about 8.5-11.5 mM, preferably about 9-11 mM, preferably about 9.5-10.5 mM, highly preferably about 10 mM RAMEB, or
about 15-25 mM RAMEB, preferably about 16-24 mM, preferably about 17-23 mM, preferably about 18-22 mM, preferably about 19-21 mM, highly preferably about 20 mM RAMEB, or
about 5-30 mM (2-Hydroxypropyl)-beta-cyclodextrin (HPBCD), preferably about 8-25 mM, more preferably about 9-22 mM HPBCD, or
about 8-12 mM HPBCD, preferably about 8.5-11.5 mM, preferably about 9-11 mM, preferably about 9.5-10.5 mM, highly preferably about 10 mM HPBCD, or
about 15-25 mM HPBCD, preferably about 16-24 mM, preferably about 17-23 mM, preferably about 18-22 mM, preferably about 19-21 mM, highly preferably about 20 mM HPBCD, or
about 5-30 mM of a mixture of RAMEB and HPBCD, preferably about 8-25 mM, more preferably about 9-22 mM of a mixture of RAMEB and HPBCD, or
about 8-12 mM of a mixture of RAMEB and HPBCD, preferably about 8.5-11.5 mM, preferably about 9-11 mM, preferably about 9.5-10.5 mM, highly preferably about 10 mM of a mixture of RAMEB and HPBCD, or
about 15-25 mM of a mixture of RAMEB and HPBCD, preferably about 16-24 mM, preferably about 17-23 mM, preferably about 18-22 mM, preferably about 19-21 mM, highly preferably about 20 mM of a mixture of RAMEB and HPBCD, and
isotonic saline and
optionally about 0.001-1 m/V % (mass/volume) BC, preferably about 0.002-0.009 m/V %, preferably about 0.003-0.009 m/V % BC, or
optionally about 0.002-0.006 m/V % BC, preferably about 0.003-0.005 m/V %, highly preferably about 0.004 m/V % BC.

Preferably the pharmaceutical composition comprises or consists of
about 800-1200 μM L-Ascorbic acid 6-palmitate (ASP), preferably about 850-1150 μM, preferably about 900-1100 μM, preferably about 950-1050 μM, highly preferably about 1000 μM ASP, and
about 8-12 mM RAMEB, preferably about 8.5-11.5 mM, preferably about 9-11 mM, preferably about 9.5-10.5 mM, highly preferably about 10 mM RAMEB, or
about 8-12 mM HPBCD, preferably about 8.5-11.5 mM, preferably about 9-11 mM, preferably about 9.5-10.5 mM, highly preferably about 10 mM HPBCD, or
about 8-12 mM of a mixture of RAMEB and HPBCD, preferably about 8.5-11.5 mM, preferably about 9-11 mM, preferably about 9.5-10.5 mM, highly preferably about 10 mM of a mixture of RAMEB and HPBCD and
isotonic saline and
optionally about 0.002-0.006 m/V % BC, preferably about 0.003-0.005 m/V %, highly preferably about 0.004 m/V % BC.

Preferably the pharmaceutical composition comprises or consists of
about 800-1200 μM L-Ascorbic acid 6-palmitate (ASP), preferably about 850-1150 μM, preferably about 900-1100 μM, preferably about 950-1050 μM, highly preferably about 1000 μM ASP, and
about 15-25 mM RAMEB, preferably about 16-24 mM, preferably about 17-23 mM, preferably about 18-22 mM, preferably about 19-21 mM, highly preferably about 20 mM RAMEB, or
about 15-25 mM HPBCD, preferably about 16-24 mM, preferably about 17-23 mM, preferably about 18-22 mM, preferably about 19-21 mM, highly preferably about 20 mM HPBCD, or
about 15-25 mM of a mixture of RAMEB and HPBCD, preferably about 16-24 mM, preferably about 17-23 mM, preferably about 18-22 mM, preferably about 19-21 mM, highly preferably about 20 mM of a mixture of RAMEB and HPBCD and
isotonic saline and
optionally about 0.002-0.006 m/V % BC, preferably about 0.003-0.005 m/V %, highly preferably about 0.004 m/V % BC.

Preferably the pharmaceutical composition comprises or consists of
about 950-1050 μM, highly preferably about 1000 μM ASP,
about 9.5-10.5 mM, highly preferably about 10 mM RAMEB, and
isotonic saline and
optionally about 0.003-0.005 m/V %, highly preferably about 0.004 n/V % BC.

Preferably the pharmaceutical composition comprises or consists of
about 950-1050 μM, highly preferably about 1000 μM ASP,
about 18-22 mM, highly preferably about 20 mM RAMEB, and
isotonic saline and
optionally about 0.003-0.005 m/V %, highly preferably about 0.004 n/V % BC.

Preferably the pharmaceutical composition comprises or consists of
about 950-1050 μM, highly preferably about 1000 μM ASP,
about 9.5-10.5 mM, highly preferably about 10 mM HPBCD, and
isotonic saline and
optionally about 0.003-0.005 m/V %, highly preferably about 0.004 n/V % BC.

Preferably the pharmaceutical composition comprises or consists of
about 950-1050 μM, highly preferably about 1000 μM ASP,
about 18-22 mM, highly preferably about 20 mM HPBCD, and
isotonic saline and
optionally about 0.003-0.005 m/V %, highly preferably about 0.004 n/V % BC.

Highly preferably the pharmaceutical composition consists of
1000 μM ASP,
10 mM RAMEB 0.004 n/V % BC, and isotonic saline.

Preferably the pharmaceutical composition consists of
1000 μM ASP,
10 mM HPBCD, 0.004 n/V % BC, and isotonic saline.

Preferably the pharmaceutical composition consists of
1000 μM ASP, −20 mM RAMEB, 0.004 n/V % BC, and isotonic saline.

Preferably the pharmaceutical composition consists of
1000 μM ASP,
20 mM HPBCD, 0.004 m/V % BC, and isotonic saline.

Preferably the isotonic saline is for pharmaceutical use, e.g. suitable for use in eye drops.

Preferably the pharmaceutical composition is lyophilized. Preferably the pharmaceutical composition is for ophthalmic (e.g. intraocular) use. Preferably the pharmaceutical composition is for use in preventing or treating corneal haze, preferably in preventing or treating corneal haze formation associated with eye surgery. Preferably the corneal haze is acute corneal haze.

Preferably the pharmaceutical composition is for use in preventing or treating fibrosis in the eye, preferably the cornea.

Preferably the pharmaceutical composition is for use in facilitating wound healing in the eye, preferably of the cornea.

Preferably the lyophilized pharmaceutical composition is to be dissolved before administration, preferably in isotonic saline. Preferably the lyophilized pharmaceutical composition is to be dissolved in e.g. 10 ml isotonic saline. Preferably the pharmaceutical composition is to be administered right after eye surgery and 1-20 times a day after the surgery, preferably 1-10 times, preferably 3-8 times, highly preferably 5 times a day. The pharmaceutical composition may be used for 30 says after surgery, or for 25 days, for 20 days, for 15 days, for 10 days or for 5 days. Preferably 1 drop is administered to one eye at a time (e.g. 1-10 times 1 drop is administered to one eye a day).

Preferably the average degree of substitution (Methyl group) is between 1.6 to 2.0 per glucose unit in RAMEB. Preferably the average degree of substitution (hydroxypropyl group) is between 2.5 to 7.0 per glucose unit in HPBCD.

### BRIEF DESCRIPTION OF THE FIGURES

FIG. 1. Solubility profiles of ASP-CD (L-Ascorbic acid 6-palmitate-cyclodextrin) complexes in case of (2-Hydroxypropyl)-gamma-cyclodextrin (HPGCD), Gamma-cyclodextrin (GCD), Sulfobutylated beta-cyclodextrin sodium salt (SBECD) (A); (2-Hydroxypropyl)-beta-cyclodextrin (HPBCD), Beta-cyclodextrin (BCD) (B) and randomly substituted Methyl-beta-cyclodextrin (RAMEB) (C).

FIG. 2. Effect of Benzalkonium chloride (BC) on solubility profiles of ASP-HPBCD (A) and ASP-RAMEB (B) complexes in physiological saline solution at 35° C.

FIG. 3. Solubility (at donor side) and corneal permeability profiles of ASP-CDs complexes in physiological saline solution at 35° C.

FIG. 4. In vitro corneal permeability values of ASP-CDs complexes at 35° C.

FIG. 5. Corneal flux profiles of ASP-CDs complexes at 35° C.

FIG. 6. Storage stability profiles of ASP-CDs complexes stored at 4° C. (A) and 25° C. (B)

FIG. 7. Relative intensity values of ASP acquired in Raman maps of concentrated formulations during 15 min treatment in comparison to ASP solution

FIG. 8. Relative intensity values of ASP acquired in Raman maps of concentrated formulations during 60 min treatment in comparison to ASP solution

FIG. 9. ASP concentration at donor site (A), in the cornea (B) and in aqueous humour (C) after treatment with 10-fold diluted formulations

FIG. 10. The flux of corneal permeability (A) and aqueous humour permeability (B) of ASP

### DETAILED DESCRIPTION OF THE INVENTION

A composition comprising ascorbic acid which effectively aids corneal wound healing process without side effects has been developed. In order to create a useful formulation, the penetrance of AA had to increased.

With higher corneal penetrance, AA-levels of deeper intraocular structures may be achieved. This may have important role in some ocular pathologies, where oxidative stress influences the pathophysiology, for example age-related macular degeneration (AMD), diabetic retinopathy or maculopathy, or glaucoma. Depletion of AA in the vitreous may be associated with macular ischemia in patients with proliferative diabetic retinopathy (18). Since AA could prevent the apoptotic loss of capillary vessel pericytes and endothelial dysfunction, it has potential role in the prevention of diabetic macular oedema (19). Oxidative stress can lead to the damage of trabecular meshwork, resulting in an increased level of intraocular pressure and loss of retinal ganglion cells, thus, it has been reported that vitamin C may influence the pathogenesis of glaucoma as well (23, 24). Increasement of the AA-level in the aqueous humour or in the vitreous may have a potential therapeutic modality of the aforementioned diseases.

Free-form AA has hydrophilic profile and acidic character; thus, permeability through the epithelium of the cornea, which provides a relatively negatively charged lipophilic lipid barrier, may be insufficient. L-Ascorbic acid 6-palmitate (ASP or 6-O-Palmitoyl-L-ascorbic acid), used as a structural analogue of AA with lipophilic properties, may be more effective in the treatment of corneal scarring due to its higher corneal permeability, however, the use of the drug alone is limited by its low local concentration due to its poor water solubility. Cyclodextrin (CD) complexations enable formulation of mainly lipophilic compositions as aqueous eye drop solution (25, 26).

Among the tested CDs, BCD, HPBCD and RAMEB showed promising aqueous solubility. However, BCD was reported extract cholesterol and other lipid components from cell membranes leading to cellular disruption and enhanced drug permeation through the corneal epithelial membrane. On the other hand, HPBCD is better tolerated in ocular tissues and less likely to cause disruption of the corneal epithelial barrier. We also found that ocular administration of RAMEB at concentrations of 5 and 12.5% was irritating to the conjunctival and corneal surface of rabbit eyes, whereas HPBCD even at a concentration of 12.5% was well tolerated.

Increasing the amount of CD had a proportional effect on the solubility of ASP, thus in order to avoid any toxicological issue, the amount of applied CDs had to be minimized in the 1-20 mM range (lower than 2.8% (m/V %).

Results from the corneal-specific parallel artificial membrane permeability assay showed that both in case of HPBCD and RAMEB 10 and 20 mM concentration of CD, beside 0.004% of BC resulted in remarkable increased donor side concentrations of ASP

Increasing the CD concentration, an inversely change can be observed in the in vitro corneal permeability, which can be claimed partly with the different donor ASP concentration and with the increased amount of applied CDs. Although cyclodextrins can enhance the penetration by increasing the solubility of drugs it has also been reported that an excess amount of cyclodextrin can lead to decrease in absorption through the cornea. The corneal permeability of ASP increases until the maximum solubility of ASP is achieved on the donor side.

### Ex Vivo Porcine Corneal Permeation Studies

It has been surprisingly found that the formulation comprising RAMEB provided superior permeation values of ASP.

The degree of substitution does not seem to affect the ability of a certain CD to increase permeability.

### Preservative:

BC is a frequently used preservative in eye drops, however, our solubility results showed that BC had a negative effect on solubility. Therefore, its concentration was minimized in the final formulation to 0.004%. This concentration is high enough to ensure required microbiological stability of the ocular product.

### Storage Conditions

Stability studies showed storage at cool place has a negative effect on the ASP-CD complexes, the concentration-time curves show the ASP content in solute phase decreases with higher tendency, however, degradation rate of ASP is accelerating by increasing the temperature. Based on this result it is preferred to store final formulation at room temperature.

The term “about” as used herein and when it refers to a concentration has the meaning usual in this field, i.e. as accurate as the method of measurement allows. The term “about” may also refer to a concentration that is in the range of 25%, preferably in the range of 20% or preferably in the range of 20% of the specific value given.

### Examples

### Materials and Methods

### Materials

L-Ascorbic acid 6-palmitate (ASP) (CAS Number: 137-66-6), benzalkonium chloride, (BC) (CAS Number: 63449-41-2), L-α-phosphatidylcholine (CAS Number: 97281-47-5) and Dulbecco's phosphate buffered saline (PBS, pH 7.4) modified, without calcium chloride and magnesium chloride, liquid, sterile-filtered, suitable for cell culture was purchased from Sigma Aldrich Co. Ltd. (Budapest, Hungary). The analytical grade solvents methanol, hexane, dodecane and chloroform were purchased from Merck KGaA (Darmstadt, Germany). Beta-cyclodextrin (BCD) (CAS Number: 7585-39-9), 2-hydroxypropyl)-beta-cyclodextrin (HPBCD) (CAS Number: 128446-35-5), gamma-cyclodextrin (GCD) (CAS Number: 17465-86-0), (2-hydroxypropyl)-gamma-cyclodextrin (HPGCD) (CAS Number: 128446-34-4), sulfobutylated beta-cyclodextrin sodium salt (SBECD) (CAS Number: 182410-00-0) and randomly substituted methyl-beta-cyclodextrin (RAMEB) (CAS Number: 128446-36-6) was kindly donated by Cyclolab Ltd. (Budapest, Hungary).

### Phase Solubility Study

Excess amounts of ASP were added to 5 mL of phosphate buffer (PBS, pH=7.4) containing increasing concentrations of CD in the 1-50 mM range in sealed glass vials. The obtained suspensions were electromagnetically stirred (500 rpm) at constant temperature (35+0.5° C.) until equilibrium (24 h). Then, aliquots were withdrawn, centrifuged using a Hermle Z323K high performance refrigerated centrifuge (Hermle AG, Goss-heim, Germany) for 30 min at 16,000 rpm to separate solid ASP crystals from ASP-CD solution and ASP concentration was assayed with HPLC-DAD.

### Development of Ocular Formulation

Excess amounts of ASP were added to 5 mL of physiological saline solutions containing increasing concentrations of CD in the 1-20 mM range in sealed glass vials. Different concentration of benzalkonium chloride (BC) (0.004 and 0.008%) as preservative agent was added to the formulation The obtained suspensions were electromagnetically stirred (500 rpm) at constant temperature (35+0.5° C.) until equilibrium (24 h). Then, aliquots were withdrawn, centrifuged using a Hermle Z323K high performance refrigerated centrifuge (Hermle AG, Goss-heim, Germany) for 30 min at 16,000 rpm to separate solid ASP crystals from ASP-CD solution and ASP concentration was assayed with HPLC-DAD.

### High Performance Liquid Chromatography (HPLC)

The determination of ASP concentration was performed with HPLC using an Agilent 1260 (Agilent Technologies, Santa Clara, USA). As stationary phase a Zorbax Eclipse© C18 column 100×4.6 mm, 5 μm (Phenomenex, Torrance, CA, USA) was applied. Isocratic elution with purified water and methanol 10:90 (v/v) was applied for 6 min at a flow rate of 1.0 mL/min at 25° C. 10 μL of the samples were injected to determine the ASP concentration. The chromatograms were detected at 255 nm using UV-VIS diode array detector. Data were evaluated using ChemStation B.04.03. Software (Agilent Technologies, Santa Clara, USA). The linear regression of the calibration line was 0.9998 the limit of detection (LOD) and quantification (LOQ) was 1.11 μg/ml and 3.33 μg/ml, respectively.

### In Vitro Corneal Permeability Measurements

Corneal-specific parallel artificial membrane permeability assay (corneal-PAMPA) was used to determine the transcorneal permeability of ASP-CD formulations. The filter donor plate (Multiscreen™-IP, MAIPN4510, pore size 0.45 μm; Millipore, Merck Ltd., Budapest, Hungary) was coated with 5 μL of phosphatidylcholine (16 mg) dissolved in 600 μL solvent mixture of 70% (v/v) hexane, 25% (v/v) dodecane and 5% (v/v) chloroform. The acceptor plate (MSSACCEPTOR; Millipore, Merck Ltd., Budapest, Hungary) was filled with 300 μL of a PBS solution of pH 7.4. 150-150 μL of the formulation and the reference solutions were applied on the membrane of the donor plate. Then, this was covered with a plate lid in order to de-crease the possible evaporation of the solvent. This sandwich system was incubated at 35° C. for 4 h (Heidolph Titramax 1000, Heidolph Instruments, Schwabach, Germany). The concentration of ASP permeated in the acceptor plate was determined using HPLC. The effective permeability of ASP was calculated using the following equation (29):

$${P}_{e} = \frac{- 2.303}{A \times ( t - {t}_{s s} )} \times ( \frac{1}{1 + {r}_{v}} ) \times lg [ - {r}_{v} + ( \frac{1 + {r}_{v}}{1 - M R} ) \times \frac{{C}_{D} ( t )}{{C}_{D} ( 0 )} ] , \qquad ( 1 )$$

where Pₑ is the effective permeability coefficient (cm/s), A is the filter area (0.3 cm²), t is the incubation time (s), τₛₛ, is the time to reach steady-state (s), rᵥ is the volume ratio of aqueous compartments (V_{D}/V_{A}), V_{D} and V_{A} are the volumes in the donor (0.15 cm³) and acceptor phase (0.3 cm³), C_{D}(t) is the concentration of the compound in the donor phase at time point t (mol/cm³), C_{D}(0) is the concentration of the compound in the donor phase at time point zero (mol/cm³) and MR is the membrane retention factor, defined as (29)

$$M R = 1 - \frac{{C}_{D} ( t )}{{C}_{D} ( 0 )} - \frac{{V}_{A} \times {C}_{A} ( t )}{{V}_{D} \times {C}_{D} ( t )} , \qquad ( 2 )$$

where C_{A}(t) is the concentration of the compound in the acceptor phase at time point t (mol/cm³). Flux (mol/cm²×s) was also calculated, using the following equation (30):

$$J = {P}_{e} \times {S}_{ASP} , \qquad ( 3 )$$

where Pₑ is the effective permeability coefficient (cm/s) and S_{ASP} is the solubility of ASP (mol/mL) in the donor phase after 4 h.

### Stability Study

Chemical stability of ASP and ASP-CD complexes was investigated in physiological saline at room condition (25±0.5° C.) and cold place condition (4±0.5° C.) for 5 days. Actual drug content was quantified using HPLC in predetermined time intervals to predict shelf-life of formulations.

### Ex Vivo Permeation Study on Porcine Cornea

The ex vivo penetration of ASP was examined with HPLC and Raman microscopy. Freshly donated porcine eyes obtained from the slaughterhouse was placed on a sterile cotton wool bed moistened with physiological saline solution and kept in refrigerator box during the transportation. The porcine eye was placed into a Teflon cell, where the cornea was uncovered and surrounded by a Teflon ring to prevent the flow of eye drops. The cornea was instilled with 1000 μL of physiological saline solution and 50 μL of ASP-CD formulation was added to the covering saline solution, which can imitate physiological dilution caused by tear fluid and incubated at 35° C. After 15, 30 and 60 min individual treatment residual solution from the topical surface and the aqueous humour was aspirated through corneal paracentesis 2-5 hours post mortem. The cornea was excised and ASP was extracted with 2 mL methanol:water 50:50 (v/v) using orbital shaker (PSU-10i Orbital Shaker, Grant Instruments Ltd, Cambs, England) for 60 min, at 450 rpm. ASP content in the residual solution, aqueous humour and cornea was determined with HPLC. The corneal retention of AS was calculated according to Eq. 2., whereas apparent permeability by using the following equation:

$${P}_{app} ( cm / s ) = \frac{{Δ [ C ]}_{A} \times {V}_{A}}{A \times {[ C ]}_{D} \times Δ t} , \qquad ( 4 )$$

where, Papp was calculated from the concentration difference of AS in the aqueous humor (Δ[C]_{A}) after treatment, initial donor concentration ([C]_{D}), the volume of anterior chamber V_{A} (250 μL) and A is the surface area available for permeability (1.77 cm²). Each measurement was performed in triplicate, data is presented as mean±SD.
Investigation of Corneal Drug Permeation with Raman Spectroscopy

Parallel to HPLC permeability determination cornea was investigated with Raman mapping after 15 and 30 min treatment. The treated cornea was frozen and divided into cross sections (15 μm thick) with a Leica CM1950 Cryostat (Leica Biosystems GmbH, Wetzlar, Germany). Aluminum-coated slides were used under the 15-μm-thick cross-sections. Raman spectroscopic analysis was carried out with a Thermo Fisher DXR Dispersive Raman Spectrometer (Thermo Fisher Scientific Inc., Waltham, MA, USA) equipped with a CCD camera and a diode laser operating at 780 nm. Microscopic lens with 50× magnification was used. Measurements were carried out with a laser power of 24 mW and a slit aperture of 50 μm. Cornea mapping was captured of an area of 150×1000 μm, with a step size of 50 μm vertically and horizontally. The OMNIC for Dispersive Raman 8.2 software (Thermo Fisher Scientific) was used for chemical evaluation. The individual spectrum of unformulated ASP was used as a reference when profiling the chemical map.

### Results

### Selection of CD Derivatives in PBS (Screening)

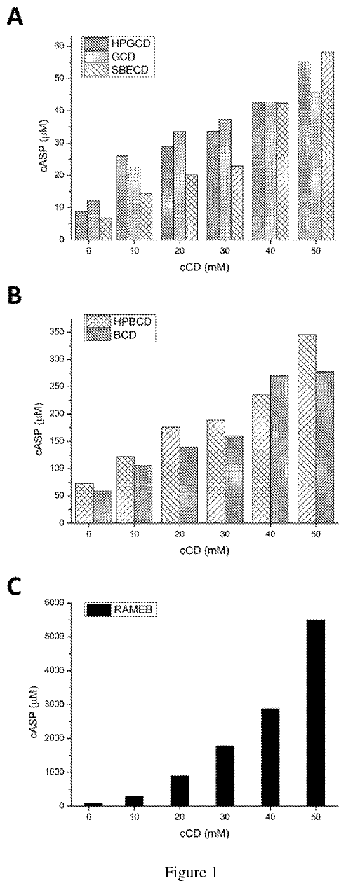

Solubility studies of ASP in presence of different concentration of CD derivatives showed enhanced solubility of ASP in PBS (pH=7.4) increasing the concentration of CD, therefore successful complexation of drug (FIG. 1.)

The solubility studies, clearly demonstrate the aqueous solubility of ASP was highest in case of complexation with BCD, HPBCD and RAMEB in comparison to GCD, HPGCD or SBECD. However, the selection of BCD was avoided, because its proven ability to extract cholesterol and other lipid components from cell membranes (31) leading to cellular disruption and enhanced drug permeation through the corneal epithelial membrane (32). Conversely, HPBCD is better tolerated in ocular tissues and less likely to cause disruption of the corneal epithelial barrier (33). Therefore, HPBCD and RAMEB were selected as suitable candidates for further investigation. However, increasing the amount of CD has a proportional effect on the solubility of ASP, in order to avoid any toxicological issue, the amount of applied CDs was minimized in 1-20 mM range (lower than 2.8%) for further investigations. Ocular administration of RAMEB at concentrations of 5 and 12.5% was irritating to the conjunctival and corneal surface of rabbit eyes, whereas HPBCD even at a concentration of 12.5% was well tolerated.

### Development of Formulations Regarding the Effect of Electrolyte and Preservatives on API Solubility

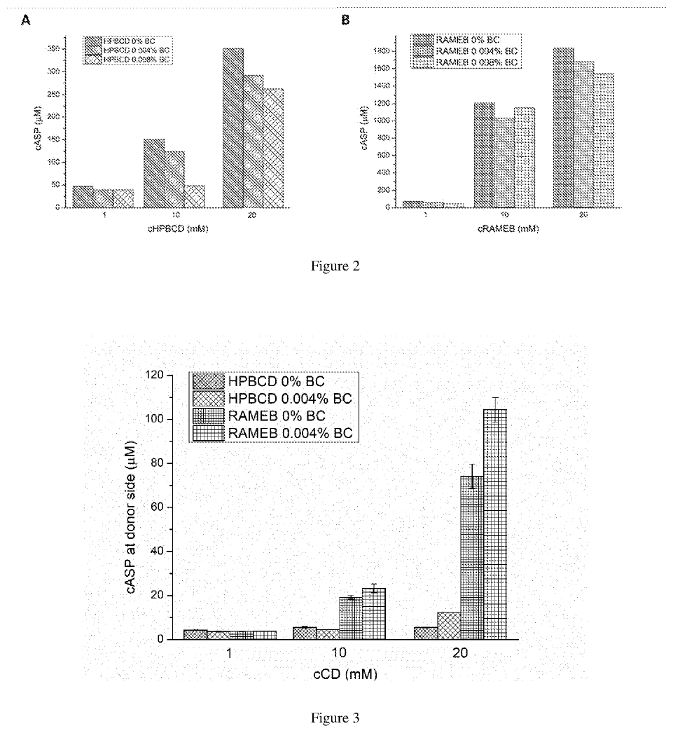

To complete the requirements of ocular formulations, PBS was changed to physiological saline solution and preservative agent was added. For that purpose, BC seemed to be most suitable, although it can also have an undesired effect on ASP solubility, namely the competition for the complexation. BC is a frequently used preservative in eye drops; typical concentrations range from 0.004 to 0.01% (35). Therefore, solubility studies were also conducted in presence of physiological saline solution and BC (0.004 and 0.008%) to elucidate it (FIG. 2.).

The results supported BC has a negative effect on solubility, moreover to minimize the chance of adverse reactions (e.g. irritation) related to BC, its concentration was minimized in the final formulation to 0.004%. This concentration is high enough to ensure required microbiological stability of the ocular product.

### In Vitro Corneal Permeability Results

Corneal-PAMPA measurement was carried out in physiological saline solution both with HPBCD and RAMEB formulations to investigate the effect of CD and BC concentration on the permeability (FIG. 3).

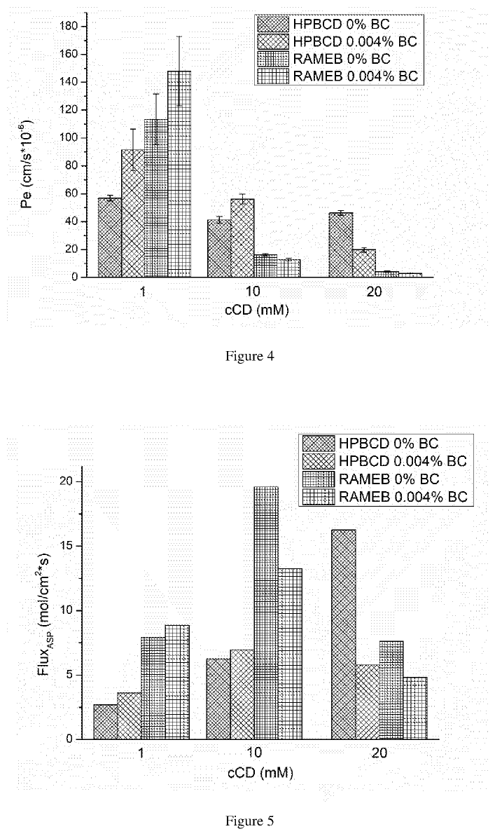

The corneal-PAMPA measurement supports the results of preformulation studies, according to which both in case of HPBCD and RAMEB 10 and 20 mM concentration of CD, beside 0.004% of BC showed remarkable increased donor side concentration of ASP. The effective permeability (FIG. 4) and flux of corneal permeation (FIG. 5) of ASP as concentration dependent factor was also calculated to elucidate which CD concentration is the most promising.

Increasing the CD concentration, an inversely change can be observed in the in vitro corneal permeability, which can be claimed partly with the different donor ASP concentration and with the increased amount of applied CDs. Although cyclodextrins can enhance the penetration by increasing the solubility of drugs it has also been reported that an excess amount of cyclodextrin can lead to decrease in absorption through the cornea. The corneal permeability of ASP increases until the maximum solubility of ASP is achieved on the donor side (36).

The flux values of corneal-PAMPA measurement of APS showed highest increase in case of 0.004% BC at 10 mM CD concentration contrary to 20 mM, which can be claimed with the different donor side concentration (5-fold) of ASP. In summary based on the corneal-PAMPA results 20 mM CD formulations seemed to be promising, therefore further characterizations were conducted with them.

### Stability Studies

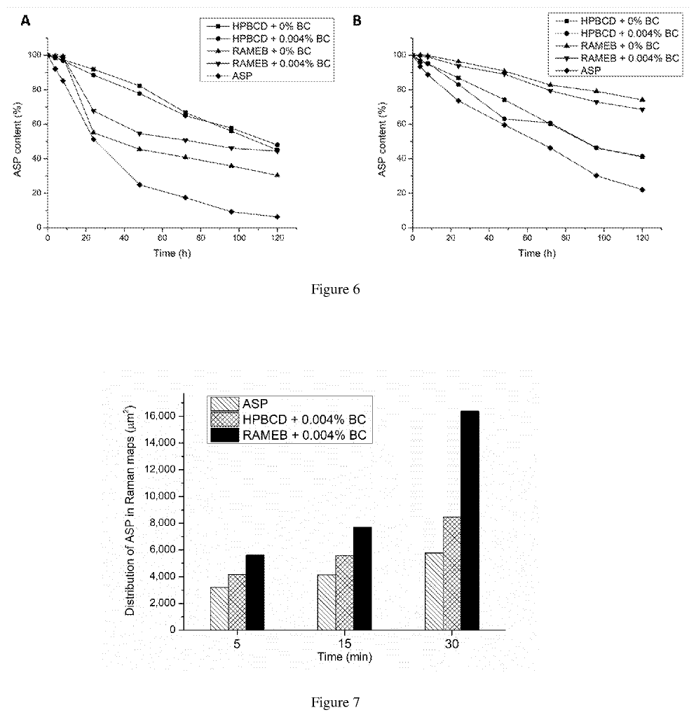

Stability studies were carried out to determine optimal storage conditions and shelf-life of ASP-CD formulations. The time-dependent changes in ASP content of different ASP-CD complexes stored at 4° C. and 25° C. are presented in FIG. 6.

Stability studies showed storage at cool place has a negative effect on the ASP-CD complexes, the concentration-time curves show the ASP content in solute phase decreases with higher tendency, however degradation rate of ASP is accelerating by increasing the temperature. This phenomenon can be claimed with the existence of saturated ASP solutions due to applied CD. By decreasing the temperature to 4° C. ASP tends to release from CD complex and precipitates in the aqueous medium, decreasing ASP concentration of liquid formulations. Based on this result it is preferred to store final formulation at room temperature. Shelf-life of ASP-CD complexes were determined based on the storage stability profiles, shown in Table 1.

TABLE 1
Shelf-life (t_{90%)} of different ASP-CD compositions

Shelf life (h)

| Composition | 4° C. | 25° C. |
|---|---|---|
| ASP | 4.58 | 8.37 |
| ASP + HPBCD + 0% BC | 15.81 | 14.16 |
| ASP + HPBCD + 0.004% BC | 17.18 | 14.25 |
| ASP + HPBCD + 0.008% BC | 16.70 | 14.49 |
| ASP + RAMEB + 0% BC | 10.57 | 42.10 |
| ASP + RAMEB + 0.004% BC | 15.55 | 33.48 |
| ASP + RAMEB + 0.008% BC | 18.21 | 33.58 |

m/v %

Shelf-life data shows CD complexes can stabilize ASP, degradation process will be sustained even 3-5 fold in comparison to initial ASP, which improves therapeutical applicability of formulations. BC shows no significant effect on the shelf-life.

### Ex Vivo Porcine Corneal Permeation Studies

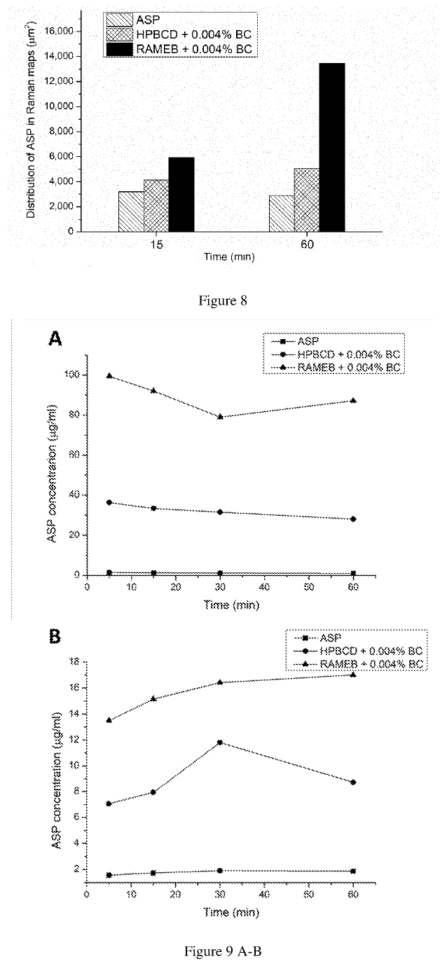

Raman mapping was carried out to examine the distribution of selected ASP-CD formulation and initial ASP on porcine cornea. Both concentrated (FIG. 7) and 10-times diluted formulations (FIG. 8) were tested to mimic physiological dilution conditions of living eye. For localization of permeated formulation, the Raman spectra of ASP was set as profile, whose frequency of occurrence was determined by measuring the relative intensity of ASP in the Raman maps with ImageJ 1.4 software (National Institutes of Health, Bethesda, MD, USA).

Raman maps after 5 min treatment clearly indicated both HPBCD and RAMEB complexes improved ocular permeation of ASP in comparison the reference ASP solution. RAMEB 0.004% BC shows higher relative intensity, which indicate higher concentration of permeated drug, compared to other two investigated samples.

After 15 min treatment the relative intensity of ASP increased also in case of ASP reference and HPBCD, indicating higher permeation rate compared to 5 min treatment. Physiologically the first 15 min is relevant for drug permeation, because of the elimination due to ocular clearance.

After 30 min treatment both HPBCD and RAMEB showed higher relative intensity in comparison to ASP solution, however in case of RAMEB remarkable higher ASP concentration can be observed.

Previous measurements were conducted also with 10-times diluted formulations to make more biorelevant the experiment, considering the physiological clearance due to tear fluid.

Diluted formulations showed similar tendency in permeation of formulations to concentrated ones, however after 15 min permeated drug still not reached the stroma. This is inessential from therapeutical view, as the cornea anticipates as site of action, its saturation represents the therapeutical aim.

After 60 min the permeation of ASP is similar to concentrated formulation, RAMEB complex shows remarkable higher relative intensity and ASP saturation in full cross-section of porcine cornea.

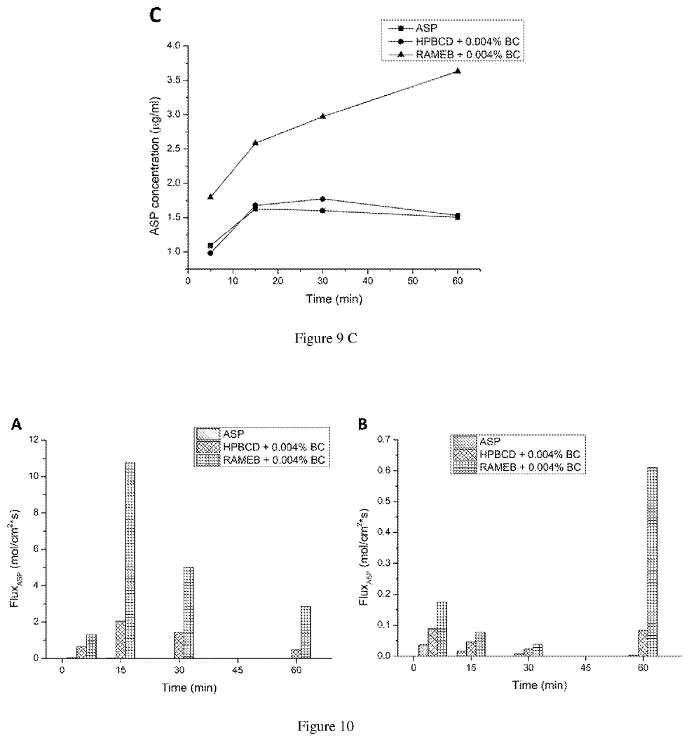

Ex vivo permeation was also investigated quantitatively by determining ASP concentration in cornea and aqueous humour using HPLC (FIG. 9).

Donor site concentrations show slight decrease in ASP concentration, which can be claimed with the increase permeation of drug into the cornea. This finding is supported with the corneal concentrations, which increase simultaneously by the time of treatment. Basically, the corneal concentrations are therapeutically important, these results predict improved therapeutical efficacy of both CD complexes. Interestingly increasing ASP concentration can be also detect in the aqueous humour, which provides large extent ocular permeation of ASP. In all cases RAMEB shows significantly increased ASP concentrations, which can be claimed with the 2.5-fold higher donor concentration to HPBCD. The flux of corneal permeability and aqueous humour permeability was also calculated (FIG. 10).

RAMEB showed remarkable increased corneal flux values in comparison to HPBCD and both CD showed significantly higher flux values to initial drug, which proves their advantageous effect on improving ocular delivery of ASP. Aqueous humour flux values showed not so unequivocal results, only a slight difference can be observed.

### REFERENCES

1. Meek K M. Corneal collagen-its role in maintaining corneal shape and transparency. Biophys Rev. 2009; 1(2):83-93. doi:10.1007/s12551-009-0011-x.
2. Mazzotta C, Hafezi F, Kymionis G, Caragiuli S, Jacob S, Traversi C, et al. In Vivo Confocal Microscopy after Corneal Collagen Crosslinking. Ocul Surf. 2015; 13:298-314. doi:10.1016/j.jtos.2015.04.007.
3. Kaji Y, Yamashita H, Oshika T. Corneal wound healing after excimer laser keratectomy. Semin Ophthalmol. 2003; 18:11-6. doi:10.1076/soph.18.1.11.14075.
4. Kim B Z, Jordan C A, McGhee C N, Patel D V. Natural history of corneal haze after corneal collagen crosslinking in keratoconus using Scheimpflug analysis. J Cataract Refract Surg. 2016; 42:1053-9. doi:10.1016/j.jcrs.2016.04.019.
5. Kwok S S, Shih K C, Bu Y, Lo A C, Chan T C, Lai J S, et al. Systematic Review on Therapeutic Strategies to Minimize Corneal Stromal Scarring After Injury. Eye Contact Lens. 2019; 45:347-55. doi:10.1097/ICL.0000000000000584.
6. Padh H. Cellular functions of ascorbic acid. Biochem Cell Biol. 1990; 68:1166-73. doi:10.1139/o90-173.
7. Chen J, Lan J, Liu D, Backman L J, Zhang W, Zhou Q, et al. Ascorbic Acid Promotes the Stemness of Corneal Epithelial Stem/Progenitor Cells and Accelerates Epithelial Wound Healing in the Cornea. Stem Cells Transl Med. 2017; 6:1356-65. doi:10.1002/sctm.16-0441.
8. Russell S B, Russell J D, Trupin K M. Collagen synthesis in human fibroblasts: effects of ascorbic acid and regulation by hydrocortisone. J Cell Physiol. 1981; 109:121-31. doi:10.1002/jcp.1041090114.
9. Pasonen-Seppanen S, Suhonen T M, Kirjavainen M, Suihko E, Urtti A, Miettinen M, et al. Vitamin C enhances differentiation of a continuous keratinocyte cell line (REK) into epidermis with normal stratum corneum ultrastructure and functional permeability barrier. Histochem Cell Biol. 2001; 116:287-97. doi:10.1007/s004180100312.
10. Hata R, Senoo H. L-ascorbic acid 2-phosphate stimulates collagen accumulation, cell proliferation, and formation of a three-dimensional tissuelike substance by skin fibroblasts. J Cell Physiol. 1989; 138:8-16. doi:10.1002/jcp.1041380103.
11. Ruggiero F, Burillon C, Garrone R. Human corneal fibrillogenesis. Collagen V structural analysis and fibrillar assembly by stromal fibroblasts in culture. Invest Ophthalmol Vis Sci. 1996; 37:1749-60.
12. Guo X, Hutcheon A E, Melotti S A, Zieske J D, Trinkaus-Randall V, Ruberti J W. Morphologic characterization of organized extracellular matrix deposition by ascorbic acid-stimulated human corneal fibroblasts. Invest Ophthalmol Vis Sci. 2007; 48:4050-60. doi:10.1167/iovs.06-1216.
13. Cho Y W, Yoo W S, Kim S J, Chung I Y, Seo S W, Yoo J M. Efficacy of systemic vitamin C supplementation in reducing corneal opacity resulting from infectious keratitis. Medicine (Baltimore). 2014; 93:e125. doi:10.1097/MD.0000000000000125.
14. Lee M Y, Chung S K. Treatment of corneal neovascularization by topical application of ascorbic acid in the rabbit model. Cornea. 2012; 31:1165-9. doi:10.1097/ICO.0b013e318241433b.
15. Kasetsuwan N, Wu F M, Hsieh F, Sanchez D, McDonnell P J. Effect of topical ascorbic acid on free radical tissue damage and inflammatory cell influx in the cornea after excimer laser corneal surgery. Arch Ophthalmol. 1999; 117:649-52. doi:10.1001/archopht.117.5.649.
16. Stojanovic A, Ringvold A, Nitter T. Ascorbate prophylaxis for corneal haze after photorefractive keratectomy. J Refract Surg. 2003; 19:338-43.
17. Singla S, Majumdar D K, Goyal S, Khilnani G. Evidence of carrier mediated transport of ascorbic acid through mammalian cornea. Saudi Pharm J. 2011; 19:165-70. doi:10.1016/j.jsps.2011.03.007.
18. Park S W, Ghim W, Oh S, Kim Y, Park U C, Kang J, et al. Association of vitreous vitamin C depletion with diabetic macular ischemia in proliferative diabetic retinopathy. PLoS One. 2019; 14:e0218433. doi:10.1371/journal.pone.0218433.
19. May J M. Ascorbic acid repletion: A possible therapy for diabetic macular edema? Free Radic Biol Med. 2016; 94:47-54. doi:10.1016/j.freeradbiomed.2016.02.019.
20. Gurreri A, Pazzaglia A, Schiavi C. Role of Statins and Ascorbic Acid in the Natural History of Diabetic Retinopathy: A New, Affordable Therapy? Ophthalmic Surg Lasers Imaging Retina. 2019; 50:S23-S7. doi:10.3928/23258160-20190108-06.
21. Arslan S, Kadayifcilar S, Samur G. The Potential Role of Dietary Antioxidant Capacity in Preventing Age-Related Macular Degeneration. J Am Coll Nutr. 2019; 38:424-32. doi:10.1080/07315724.2018.1538830.
22. Sant D W, Camarena V, Mustafi S, Li Y, Wilkes Z, Van Booven D, et al. Ascorbate Suppresses VEGF Expression in Retinal Pigment Epithelial Cells. Invest Ophthalmol Vis Sci. 2018; 59:3608-18. doi:10.1167/iovs.18-24101.
23. Ramdas W D, Schouten J, Webers C A B. The Effect of Vitamins on Glaucoma: A Systematic Review and Meta-Analysis. Nutrients. 2018; 10. doi:10.3390/nu10030359.
24. Xu P, Lin Y, Porter K, Liton P B. Ascorbic acid modulation of iron homeostasis and lysosomal function in trabecular meshwork cells. J Ocul Pharmacol Ther. 2014; 30:246-53. doi:10.1089/jop.2013.0183.
25. Johannsdottir S, Jansook P, Stefansson E, Loftsson T. Development of a cyclodextrin-based aqueous cyclosporin A eye drop formulations. Int J Pharm. 2015; 493:86-95. doi:10.1016/j.ijpharm.2015.07.040.
26. Jansook P, Pichayakorn W, Muankaew C, Loftsson T. Cyclodextrin-poloxamer aggregates as nanocarriers in eye drop formulations: dexamethasone and amphotericin B. Drug Dev Ind Pharm. 2016; 42:1446-54. doi:10.3109/03639045.2016.1141932.
27. Johannesson G, Moya-Ortega M D, Asgrimsdottir G M, Lund S H, Thorsteinsdottir M, Loftsson T, et al. Kinetics of gamma-cyclodextrin nanoparticle suspension eye drops in tear fluid. Acta Ophthalmol. 2014; 92:550-6. doi:10.1111/aos.12334.
28. Loftssona T, Jarvinen T. Cyclodextrins in ophthalmic drug delivery. Adv Drug Deliv Rev. 1999; 36:59-79. doi:10.1016/s0169-409x(98)00055-6.
29. Avdeef, A. Permeability-PAMPA Absorption and Drug Development 2012:319-498.
30. Dargó G, Vincze A, Müller J, Kiss H J, Nagy Z Z, Balogh G T. Corneal-PAMPA: A novel, non-cell-based assay for prediction of corneal drug permeability. Eur. J. Pharm. Sci. 2019; 128:232-239. doi: 10.1016/j.ejps.2018.12.012.
31. Ohtani Y, Irie T, Uekama K, Fukunaga K, Pitha J. Differential effects of α-, β- and γ-cyclodextrins on human erythrocytes. Eur. J. Biochem. 1989; 186:17-22. doi: 10.1111/j.1432-1033.1989.tb15171.x.
32. Morrison W, Connon C J, Khutoryanskiy V. Cyclodextrin-mediated enhancement of riboflavin solubility and corneal permeability. Mol. Pharm. 2013; 10:756-762. doi: 10.1021/mp3005963
33. European Medicines Agency, Background Review for Cyclodextrins Used as Excipients, 2014:1-17.
34. Cyclodextrin News, 2013; 27:2.
35. Choi H Y, Lee Y H, Lim C H, Kim Y S, Lee I S, Jo J M, Ha Y L, Hyo G C, Hee J W, Seo D S. Assessment of respiratory and systemic toxicity of Benzalkonium chloride following a 14-day inhalation study in rats. Part. Fibre. Toxicol. 2020; 17:1-19. doi: 10.1186/s12989-020-0339-8
36. Másson M, Loftsson T, Másson G, Stefánsson E. Cyclodextrins as permeation enhancers: some theoretical evaluations and in vitro testing. J. Control. Rel. 1999; 59:107-118. doi: 10.1016/s0168-3659(98)00182-5.

1-15. (canceled)

16. Pharmaceutical composition comprising L-ascorbic acid 6-palmitate (ASP), a beta-cyclodextrin selected from the group consisting of random methylated beta-cyclodextrin (RAMEB), (2-hydroxypropyl)-beta-cyclodextrin (HPBCD) and mixtures thereof, and isotonic saline.

17. The pharmaceutical composition according to claim 16, further comprising benzalkonium-chloride (BC).

18. The pharmaceutical composition according to claim 17, wherein the pharmaceutical composition consists of ASP, a beta-cyclodextrin selected from the group consisting of RAMEB and HPBCD, isotonic saline and BC.

19. The pharmaceutical composition according to claim 16, comprising
about 800-1200 μM ASP and
about 5-30 mM RAMEB or
about 8-12 mM RAMEB or
about 15-25 mM RAMEB or
about 5-30 mM (2-Hydroxypropyl)-beta-cyclodextrin (HPBCD), or
about 8-12 mM HPBCD or
about 15-25 mM HPBCD and
isotonic saline.

20. The pharmaceutical composition according to claim 19, comprising
about 800-1200 μM L-Ascorbic acid 6-palmitate (ASP) and
about 8-12 mM RAMEB or
about 8-12 mM HPBCD and
isotonic saline.

21. The pharmaceutical composition according to claim 20, comprising
about 950-1050 μM ASP,
a beta-cyclodextrin selected from about 9.5-10.5 mM RAMEB, about 9.5-10.5 mM HPBCD, and
isotonic saline.

22. The pharmaceutical composition according to claim 19, comprising
about 950-1050 μM ASP,
a beta-cyclodextrin selected from about 18-22 mM RAMEB, about 18-22 mM HPBCD, and
isotonic saline.

23. The pharmaceutical composition according to claim 16, consisting of
about 1000 μM ASP,
about 10 mM RAMEB,
about 0.004 m/V % BC, and
isotonic saline.

24. The pharmaceutical composition according to claim 16, consisting of
about 1000 μM ASP,
about 10 mM HPBCD,
about 0.004 m/V % BC, and
isotonic saline.

25. The pharmaceutical composition according to claim 16, consisting of
about 1000 μM ASP,
about 20 mM RAMEB,
about 0.004 m/V % BC, and
isotonic saline.

26. The pharmaceutical composition according to claim 16, consisting of
about 1000 μM ASP,
about 20 mM HPBCD,
about 0.004 m/V % BC, and
isotonic saline.

27. The pharmaceutical composition according to claim 16, wherein the pharmaceutical composition is lyophilized.

28. A method for the prevention or treatment of a disorder in the eye, the method comprising administering the pharmaceutical composition according to claim one to a patient in need thereof.

29. The method according to claim 28, wherein the disorder in the eye is selected from fibrosis, preferably fibrosis in the cornea, corneal haze, wound.
